(19) United States
(12) Patent Application Publication
(10) Pub. No.: US 2017/0007337 A1
(43) Pub. Date: Jan. 12, 2017

(54) DRIVER-MOUNTED TORQUE SENSING MECHANISM

(71) Applicant: Auris Surgical Robotics, Inc., San Carlos, CA (US)

(72) Inventor: Yoichiro Dan, Mountain View, CA (US)

(21) Appl. No.: 15/191,411

(22) Filed: Jun. 23, 2016

Related U.S. Application Data
(60) Provisional application No. 62/190,179, filed on Jul. 8, 2015.

(51) Int. Cl. A61B 34/30; A61B 17/00; A61B 34/00

(52) U.S. Cl. CPC A61B 34/30; A61B 34/71; A61B 17/00234; A61B 2034/302; A61B 2017/00323; A61B 2034/301; A61B 2017/00398

(57) ABSTRACT

A robotically-controlled drive unit includes a torque sensing mechanism to measure the torque applied to a rotatable body that is configured to tension an actuation tendon to operate robotic surgical tools and catheters. The drive unit includes a motor unit that generates an output torque in response to a robotic control signal. A beam element generates a reactive torque in response to the output torque generated by the rotor, and a force sensor detects the reactive torque and communicates the magnitude of the reactive torque to a robotic controller. The drive unit may further include a mechanism to perform bi-directional torque sensing, examples of which include additional force sensors and compression springs.

### CROSS-REFERENCE TO RELATED APPLICATIONS

This application claims the benefit of and priority to U.S. Provisional Application No. 62/190,179 filed Jul. 8, 2015, the entire contents of which are incorporated herein by reference. This application is related to U.S. patent application Ser. No. 14/523,760, filed Oct. 24, 2014, U.S. Provisional Patent Application No. 62/019,816, filed Jul. 1, 2014, U.S. Provisional Patent Application No. 62/037,520, filed Aug. 14, 2014, U.S. Provisional Patent Application No. 62/057,936, filed Sep. 30, 2014, U.S. Provisional Patent Application No. 62/134,366, filed Mar. 17, 2015, the entire contents of which are incorporated herein by reference.

### BACKGROUND

1. Field of Art

This description relates to a driver-mounted tension sensing design that may be used in conjunction with a medical robotics platform for a number of surgical procedures. More particularly, the driver-mounted tension sensing mechanism detects torques exerted by output shafts within an instrument device manipulator that actuates tendons to operate an elongated instrument.

2. Description of Related Art

Use of robotic technologies presents a number of advantages over traditional, manual surgery procedures. In particular, robotic surgeries often allow for greater precision, control, and access. Robotically-controlled technologies, however, sometimes create engineering challenges that require creative engineering workarounds. In the case of robotically-controlled tools, the use of actuation tendons to operate robotic laparoscopic tools and catheters gives rise to control problems that often requires very precise monitoring of the torque applied to actuation tendons. Over the lifespan of an actuation tendon, the tendon may stretch and deform, and exhibit greater non-linearity with respect to force applied to the tendon and the expected actuation. Accordingly, within a robotically-controlled instrument, there is a need to accurately measure the torque applied to a rotatable body.

### SUMMARY

In general, the present description provides a medical device that includes a robotically-controlled drive unit configured to generate angular motion, the drive unit including an electric motor unit with a rotor that is configured to generate an output torque in response to a robotic control signal, a beam element configured to generate a reactive torque in response to the output torque generated by the rotor, and a force sensor configured to detect the reactive torque and communicate the magnitude of the reactive torque to a robotic controller, wherein the beam element is coupled to the electric motor unit and oriented perpendicularly to the longitudinal axis of the rotor.

In one aspect, the beam element is configured to rotate around the axis of the motor shaft using a ball bearing. In one aspect, the beam element is coupled to a rotatable component that is configured to rotate around the axis of the rotor using a ball bearing.

In one aspect, the drive unit further includes a gear head coupled to the electric motor unit, wherein the gear head is configured to amplify the output torque generated by the rotor. In another aspect, the drive unit further includes a drive base that includes a ball bearing configured to allow the beam element to move freely in response to the output torque. In another aspect, the drive unit further includes an output shaft operatively coupled to the gear head. In another aspect, the output shaft is configured to transmit angular motion to a rotatable body intended to actuate a tendon in an elongated body. In another aspect, the elongated body is an instrument configured for performing endolumenal procedures. In another aspect, the elongated body is a flexible instrument. In another aspect, the elongated body is a catheter. In another aspect, the elongated body is an instrument configured for performing laparoscopic procedures. In another aspect, the output shaft is configured to rotate an elongated body along its longitudinal axis.

In one aspect, the force sensor includes at least one of a load cell, piezoresistive device, a piezoelectric device, and a strain gauge. In one aspect, the drive unit further includes a stopper that is configured to keep the beam element from moving in the direction of the stopper. In another aspect, the stopper and the force sensor are positioned on opposite sides of the beam element. In another aspect, the stopper and the force sensor are coupled to a drive base.

In one aspect, the drive unit is incorporated into an instrument device manipulator as part of a surgical robotics platform. In one aspect, the instrument device manipulator includes a second drive unit. In one aspect, the robotic controller is remotely located. In one aspect, the robotic controller is configured to generate the robotic control signal. In one aspect, the robotic controller is configured to generate the robotic control signal based on the magnitude of the reactive torque detected by the force sensor. In one aspect, the drive unit includes a rotary encoder coupled to the electric motor unit and configured to detect angular motion of the motor shaft.

### BRIEF DESCRIPTION OF DRAWINGS

FIG. 1A illustrates a perspective view of a drive unit configured to generate angular motion that incorporates a torque sensing mechanism, consistent with one embodiment.

FIG. 1B illustrates a top view of the drive unit from FIG. 1A, consistent with one embodiment.

FIG. 1C illustrates a side view of the drive unit from FIGS. 1A and 1B, consistent with one embodiment.

FIG. 1D illustrates a perspective view of a drive unit configured to generate angular motion, and that incorporates a bi-directional torque sensing mechanism with two force sensors, consistent with one embodiment.

FIG. 1E illustrates a top view of the drive unit from FIG. 1D, consistent with one embodiment.

FIG. 1F illustrates a top view of a drive unit configured to generate angular motion, and that incorporates a bi-directional torque sensing mechanism with compression springs, consistent with one embodiment.

FIG. 2A illustrates a perspective view of a plurality of drive units as configured to be used in an instrument device manipulator, consistent with one embodiment.

FIG. 2B illustrates a top view of an instrument device manipulator from FIG. 2A, consistent with one embodiment.

FIGS. 2C and 2D illustrate alternative side perspectives on the instrument device manipulator from FIGS. 2A and 2B.

FIG. 3A illustrates one view of the alignment of an instrument device manipulator in combination with an associated instrument, consistent with one embodiment.

FIG. 3B illustrates another view of the alignment of an instrument device manipulator in combination with an associated instrument, consistent with one embodiment.

FIG. 4 illustrates an instrument drive mechanism that incorporates a torque sensing mechanism, consistent with one embodiment.

FIG. 5A illustrates the incorporation of a drive unit with a bi-directional torque sensing mechanism into an instrument device manipulator with co-axial output shafts, consistent with one embodiment.

FIG. 5B illustrates the incorporation of a drive unit with a bi-directional torque sensing mechanism into an instrument device manipulator with co-axial output shafts from an another perspective, consistent with one embodiment.

FIG. 5C illustrates the incorporation of a drive unit with a bi-directional torque sensing mechanism into an instrument device manipulator with co-axial output shafts from an yet another alternative perspective, consistent with one embodiment.

FIG. 5D illustrates the incorporation of a drive unit with a bi-directional torque sensing mechanism into an instrument device manipulator with co-axial output shafts from yet another alternative perspective, consistent with one embodiment.

### DETAILED DESCRIPTION

Although certain preferred embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components.

To guarantee control fidelity, it may be important to monitor the tendon tension when robotically-controlling endoscopic and laparoscopic instruments that use tendon-like members. While there are a number of approaches to monitoring tendon tension, tension measurement in the instrument driver provides a number of practical advantages, including reduction of cost in the instrument. This is particularly important where the instruments are intended to be disposable, or “reposable,” over time. Accordingly, the present description provides a sensing apparatus that may be mounted within the instrument driver.

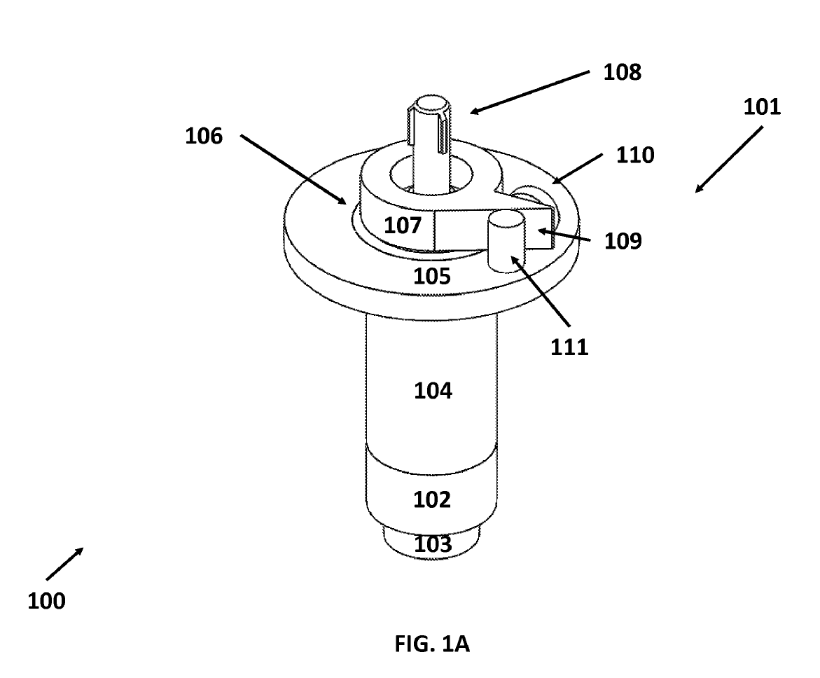

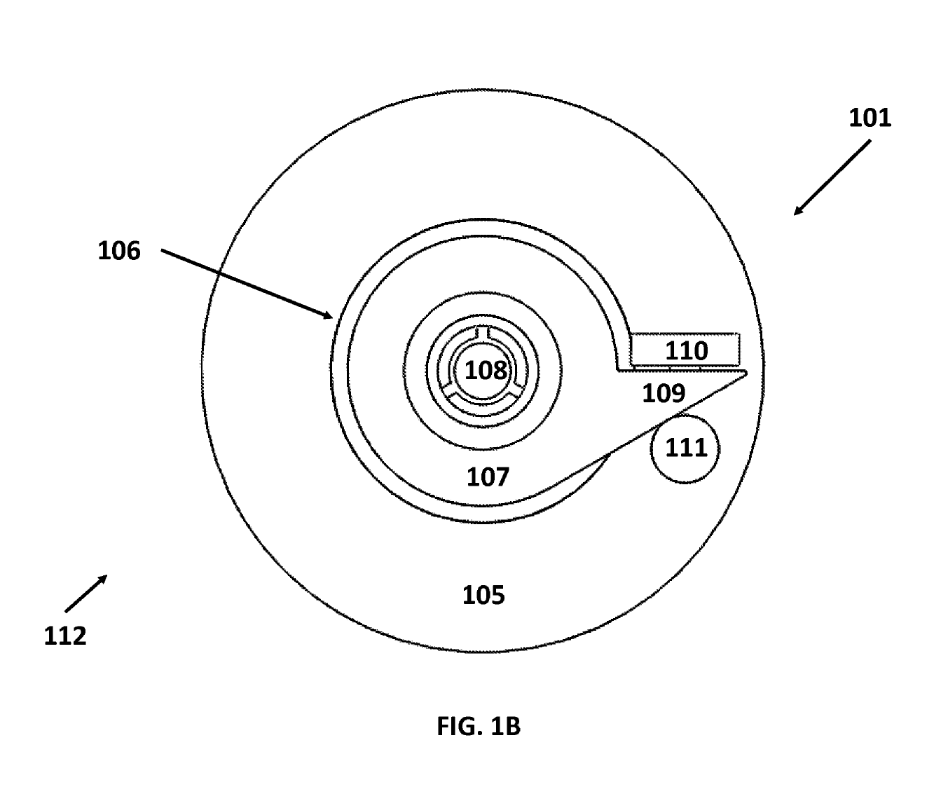

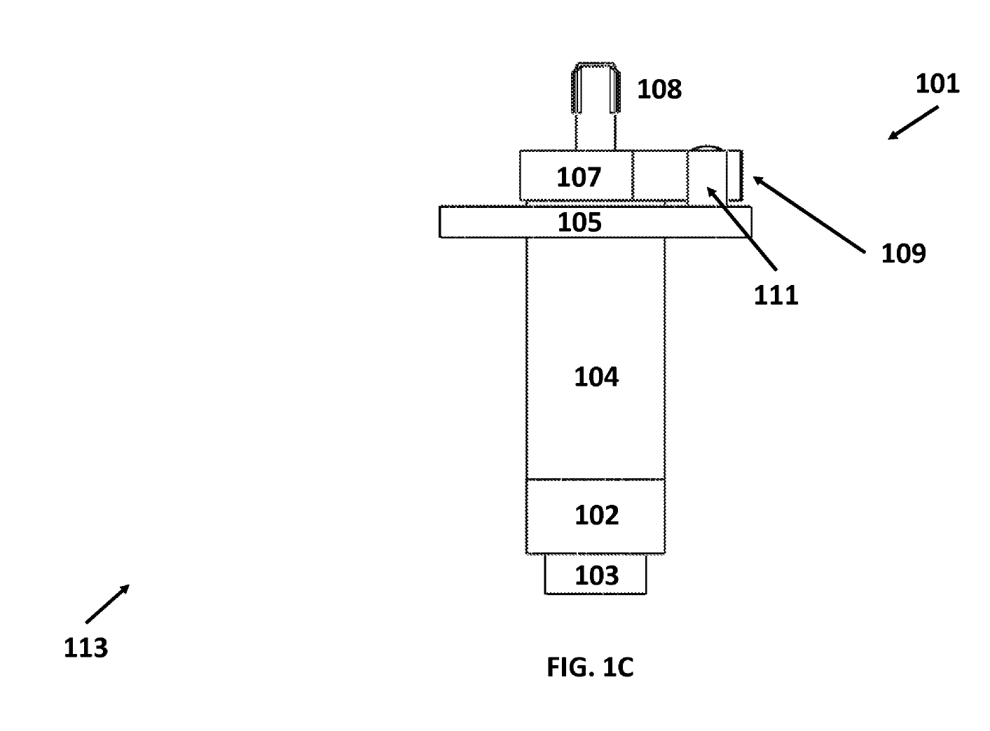

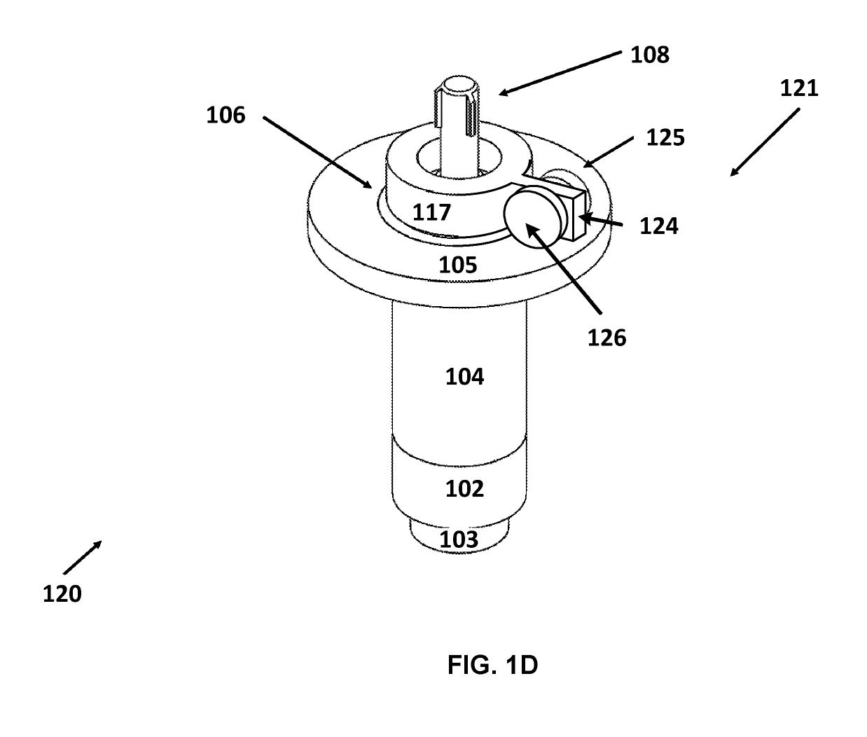

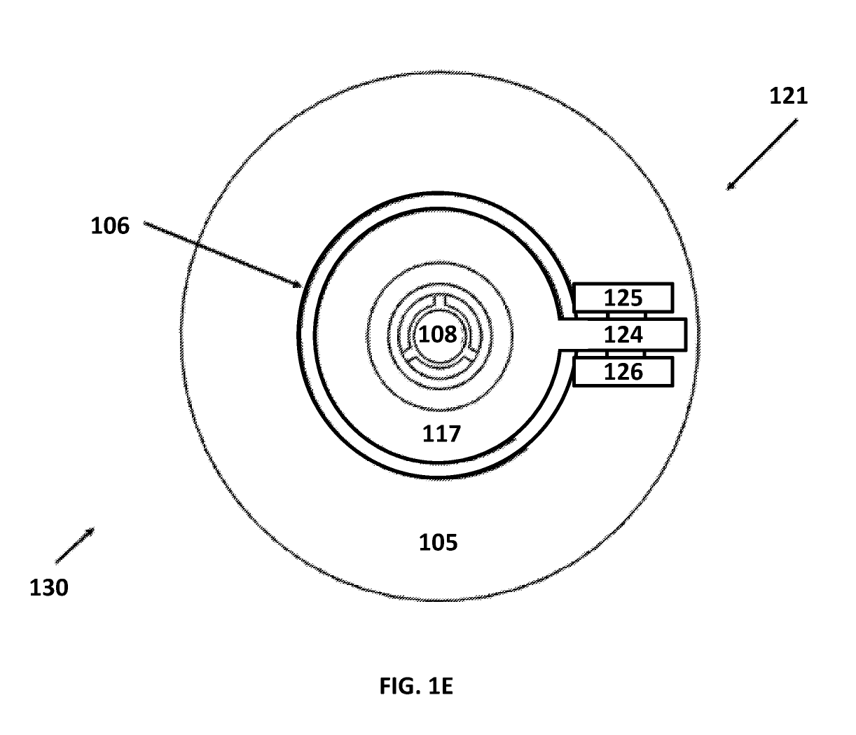

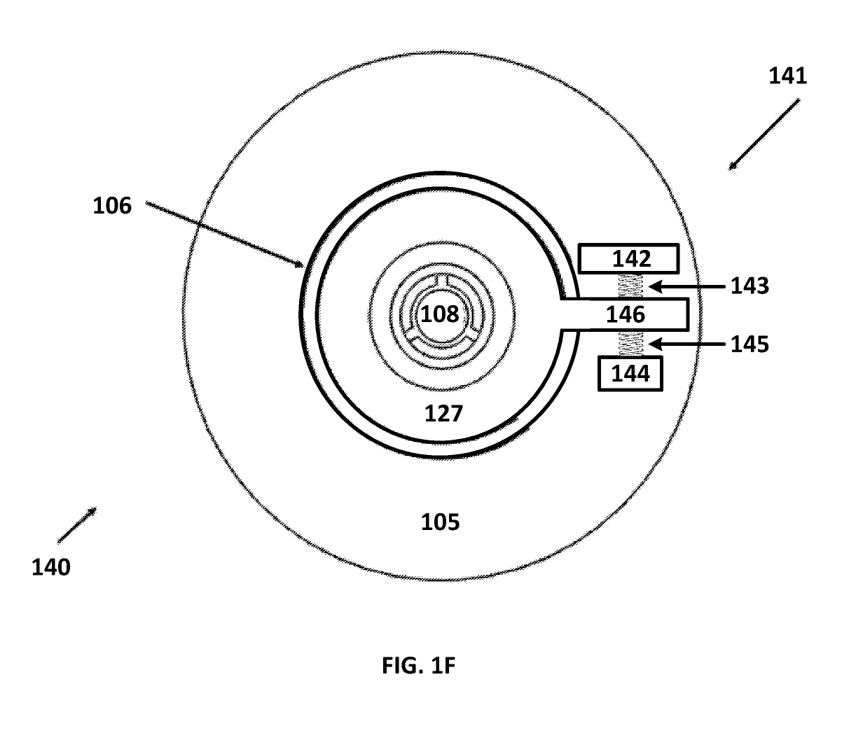

FIG. 1A illustrates a perspective view of a drive unit configured to generate angular motion that incorporates a torque sensing mechanism, consistent with one embodiment.

As shown in isometric view 100, a drive unit 101 may generally include a motor unit 102, a rotary encoder 103, and a gear head 104, and a drive base 105. The motor unit 102 may include a motor, such as a brushed or brushless motor or other type of electric motor.

Rotary encoder 103 monitors and measures the angular speed of the driveshaft of motor unit 102. In some embodiments, rotary encoder 103 may be a redundant rotary encoder. The structure, capabilities, and use of an appropriate redundant encoder is disclosed in U.S. Provisional Patent Application No. 62/037,520, filed Aug. 14, 2014, the entire contents of which are incorporated by reference.

Within a larger robotic system, a control signal from a controller component may trigger angular motion in the motor unit 102. The resulting output torque generated by the motor 102 may be transmitted to gear head 104 through a shaft coupled to the rotor of motor 102. Drive base 105 may include a bearing 106 that may be any type of low-friction bearing, such as a ball bearing and a roller bearing. In the alternative, the bearing 106 may be switched for a bushing or any other mechanical component that allows for angular motion. In some embodiments, the gear head 104 may be attached to the motor 102 to increase torque of the motor output, at the cost of the rotational speed. The increased torque generated by gear head 104 may be transmitted into gear head shaft 108. The gear head shaft 108 may include a series of splines configured to interface with rotatable bodies, such as pulleys, that may be used to tension tendons and articulate an instrument having an elongated body. Other embodiments may use alternative engagement structures, such as pegs, indentations, etc. In some embodiments, a gear head 104 may be unnecessary and thus omitted.

A rotary component 107 may be coupled to the gear head 104 and motor unit 102. Thus, any reactive angular motion in gear head 104 and motor unit 102 generated in response to torque in the rotor of motor unit 102 (and thus gear head shaft 108) may be reflected in substantially identical angular motion in rotary component 107. The rotary component 107 may be configured to freely rotate on a circular interface with bearing 106 in the drive base 105. The use of a ball bearing is intended to provide a frictionless or near-frictionless path for the rotary component 107 to rotate. In some embodiments, the circular interface may be co-axial with the gear head shaft 108, resulting a co-axial path for rotation of the rotary component 107 around the gear head shaft 108.

Designed to wrap around gear head shaft 108, the rotary component 107 may be configured to freely rotate in the in response to angular motion (and thus torque) of gear head shaft 108. As discussed earlier, in some embodiments, the rotary component 107 may be operatively coupled to the gear head 104 and motor unit 102 such that the rotary component rotates consistent with angular motion in the motor unit 102 or gear head 104 that may be generated in response to torque from the rotor of the motor unit 102 or gear head shaft 108.

The rotary component 107 may include a beam element 109 that may be configured to exert a (reactive) force on a sensor 110 in response to clockwise angular motion and torque of gear head shaft 108. For example, as shown in view 100, beam element 109 may protrude horizontally from the rotary component 107 and orthogonal to the axis of the gear head shaft 108.

In practice, clockwise angular motion, and the resulting torque, by gear head shaft 108 generates rotational motion in the counter-clockwise direction for rotary component 107. As beam element 109 may be fixedly coupled to the rotary component 107, its counter-clockwise motion around the axis of the gear head shaft 108 results in the beam element 109 exerting force on the force sensor 110. In some embodiments, the reactive force on the force sensor 110 is proportional to the torque of the gear head shaft 108. Force sensor 110 may be any sensor that can detect applied force from the beam element 109, such as a load cell, piezoresistive device, a piezoelectric device, or a strain gauge. Drive unit 101 may also include a stopper 111 to prevent angular motion in the opposite direction of force sensor 110, i.e., in the same direction as the torque of the gear shaft head 108. In some embodiments, the stopper 111 or force sensor 110 may be fixedly coupled to the base drive 105.

The stopper, force sensor, and drive base may be fixed relative to each other. For example, in some embodiments, the base may be anchored or coupled to a single rigid frame, base, or mount. In addition, the force sensor may be “pre-loaded” by positioning the stopper against the beam element, such that the force sensor detects a force measurement, e.g., a “baseline”, even when there is no angular motion in the gear head shaft 108. When torque occurs and force is applied against the force sensor, the force measurement may increase. If torque is applied in the opposite direction, the force measurement may decrease. This allows for a single force sensor to detect angular motion in both clockwise and counter-clockwise directions. An example embodiment that detects angular motion in both clockwise and counter-clockwise directions is discussed below with respect to FIG. 1F.

In some embodiments, the base 105 may be coupled to an exterior shell, electronic components, or other mechanisms within an instrument drive mechanism. A base may also stabilize drive unit 101 within an instrument device manipulator within a larger robotic system. In some embodiments, the drive base (or coupled motor mounts) may be constructed from aluminum to reduce weight.

Torque in the gear head shaft 108 may be used to tension tendons within an attached instrument. The instrument may incorporate an elongate body, such as an endoscope or a catheter, which incorporates tendons, such as pull wires or cables, to actuate or articulate a distally located end effector or a distal end.

FIG. 1B illustrates a top view of the drive unit from FIG. 1A, consistent with one embodiment. As shown in top view 112, rotary component 107 may be concentric with the axis of the gear head shaft 108. Additionally, beam element 109 may extend from rotary component 107 and may be shaped to exert force on force sensor 110 as the rotary component 107 (and the coupled gear head and motor unit) rotates in a counter-clockwise fashion in response to clockwise motion of the gear head shaft 108.

Although drive unit 101 may be configured with stopper 111 to prevent clockwise motion by rotary component 107 and beam element 109, in some embodiments, the drive unit may be configured to detect reactive force from the beam sensor in both directions. For example, in some embodiments, the stopper 111 may be replaced with a second force sensor that is configured to detect reactive force from beam element 109 when rotary component 107 (and the coupled gear head and motor unit) rotates in a clockwise fashion in response to counter-clockwise angular motion from the gear head shaft 108. An example embodiment of the drive unit that has a second force sensor is discussed below with respect to FIGS. 1D-1E.

FIG. 1C illustrates a side view of the drive unit from FIGS. 1A and 1B, consistent with one embodiment. As shown in side view 113, drive unit 101 is oriented such that the proximal (bottom) end of the motor unit 102 is coupled to a rotary encoder 103 while the distal (top) end of the motor unit 102 is coupled to a gear head 104 with a gear head shaft 108 that ultimately derives torque from the rotor of the motor unit 102. Rotary component 107 may be coupled to a ball bearing interface (106) that allows the rotary component 107 to freely rotate relative to base 105 and around the gear head shaft 108.

Beam element 108 may extend and protrude from rotary component 107 and may be shaped to exert force on force sensor 110 as the rotary component 107 rotates in a counter-clockwise fashion in response to clockwise motion of the gear head shaft 108.

FIG. 1D illustrates a perspective view of a drive unit configured to generate angular motion, and that incorporates a bi-directional torque sensing mechanism with two force sensors, consistent with one embodiment. As shown in isometric view 120, a drive unit 121 may generally include a motor unit 102, a rotary encoder 103, and a gear head 104, and a drive base 105, as discussed above with respect to FIGS. 1A-1C.

Similar to the rotary component 107 of FIGS. 1A-1C, a rotary component 117 may be coupled to the gear head 104 and motor unit 102. The rotary component 117 may include a beam element 124 that may be configured to exert a (reactive) force on sensors 125 and 126 in response to angular motion and torque of gear head shaft 108. For example, as shown in view 120, beam element 124 may protrude horizontally from the rotary component 117 and orthogonal to the axis of the gear head shaft 108.

In practice, clockwise angular motion, and the resulting torque, by gear head shaft 108 generates rotational motion in the counter-clockwise direction for rotary component 117. As beam element 124 may be fixedly coupled to the rotary component 117, its counter-clockwise motion around the axis of the gear head shaft 108 results in the beam element 124 exerting force on the force sensor 125. Conversely, counter-clockwise angular motion, and the resulting torque, by gear head shaft 108 generates rotational motion in the clockwise direction for rotary component 117. Accordingly, the clockwise motion of the beam element 124 around the axis of the gear head shaft 108 results in the beam element 124 exerting force on the force sensor 126.

In some embodiments, the reactive force on the force sensors 125, 126 is proportional to the torque of the gear head shaft 108. Force sensors 125, 126 may be any sensor that can detect applied force from the beam element 124, such as a load cell, piezoresistive device, a piezoelectric device, or a strain gauge.

The force sensors 125, 126 and drive base 105 may be fixed relative to each other. For example, in some embodiments, the base may be anchored or coupled to a single rigid frame, base, or mount.

FIG. 1E illustrates a top view of the drive unit from FIG. 1D, consistent with one embodiment. As shown in top view 130, rotary component 117 may be concentric with the axis of the gear head shaft 108. Additionally, beam element 124 may extend from rotary component 117 and may be shaped to exert force on force sensors 125, 126 as the rotary component 117 (and the coupled gear head and motor unit) rotates in response to motion of the gear head shaft 108.

FIG. 1F illustrates a top view of a drive unit configured to generate angular motion, and that incorporates a bi-directional torque sensing mechanism with compression springs, consistent with one embodiment. A drive unit 141 may generally include a motor unit, a rotary encoder, and a gear head, and a drive base 105, as discussed above with respect to FIGS. 1A-1C.

Similar to the rotary components 107, 117 of FIGS. 1A-1E, a rotary component 127 may be coupled to the gear head and motor unit. The rotary component 127 may include a beam element 146 that may be configured to exert a (reactive) force, via a compression spring 143, on sensor 142 in response to angular motion and torque of gear head shaft 108. For example, the beam element 146 may protrude horizontally from the rotary component 127 and orthogonal to the axis of the gear head shaft 108. The beam element may be coupled to the force sensor 142 via a compression spring 143 such that rotation of the rotary component 127 causes the beam element 146 to compress or stretch the compression spring 143, whereby exerting a force on the force sensor 142. The beam element may further be coupled to a stopper 144 via a compression spring 145 such that the compression spring 145 exerts a similar force on the beam element 146 as the compression spring 143 when there is no angular motion in the gear head shaft 108. The force sensor 142, stopper 144, and drive base 105 may be fixed relative to each other. For example, in some embodiments, the base may be anchored or coupled to a single rigid frame, base, or mount. As a result, the force sensor 142 may be “pre-loaded” such that the force sensor detects a force measurement, e.g., a “baseline”, even when there is no angular motion in the gear head shaft 108. When torque occurs and force is applied against the force sensor, the force measurement may increase. In case with torque in the opposite direction, the force measurement may decrease. This allows for a single force sensor to detect angular motion in both clockwise and counter-clockwise directions.

In practice, clockwise angular motion, and the resulting torque, by gear head shaft 108 generates rotational motion in the counter-clockwise direction for rotary component 127. As beam element 146 may be fixedly coupled to the rotary component 127, its counter-clockwise motion around the axis of the gear head shaft 108 causes the beam element 146 to compress the compression spring 143, which results in an increase in the force exerted by the compression spring 143 on the force sensor 142. Conversely, counter-clockwise angular motion, and the resulting torque, by gear head shaft 108 generates rotational motion in the clockwise direction for rotary component 127. Accordingly, the clockwise motion of the beam element 146 around the axis of the gear head shaft 108 causes the beam element 146 to decompress the compression spring 143, which results in a decrease in the force exerted by the compression spring 143 on the force sensor 126.

In some embodiments, the reactive force on the force sensor 142 is proportional to the torque of the gear head shaft 108. Force sensor 142 may be any sensor that can detect applied force from the beam element 146 or compression spring 143, such as a load cell, piezoresistive device, a piezoelectric device, or a strain gauge.

As shown in top view 140, the rotary component 127 may be concentric with the axis of the gear head shaft 108. Additionally, beam element 146 may extend from rotary component 127 and may be shaped to exert force on force sensor 142 via compression springs 143 as the rotary component 127 (and the coupled gear head and motor unit) rotates in response to motion of the gear head shaft 108.

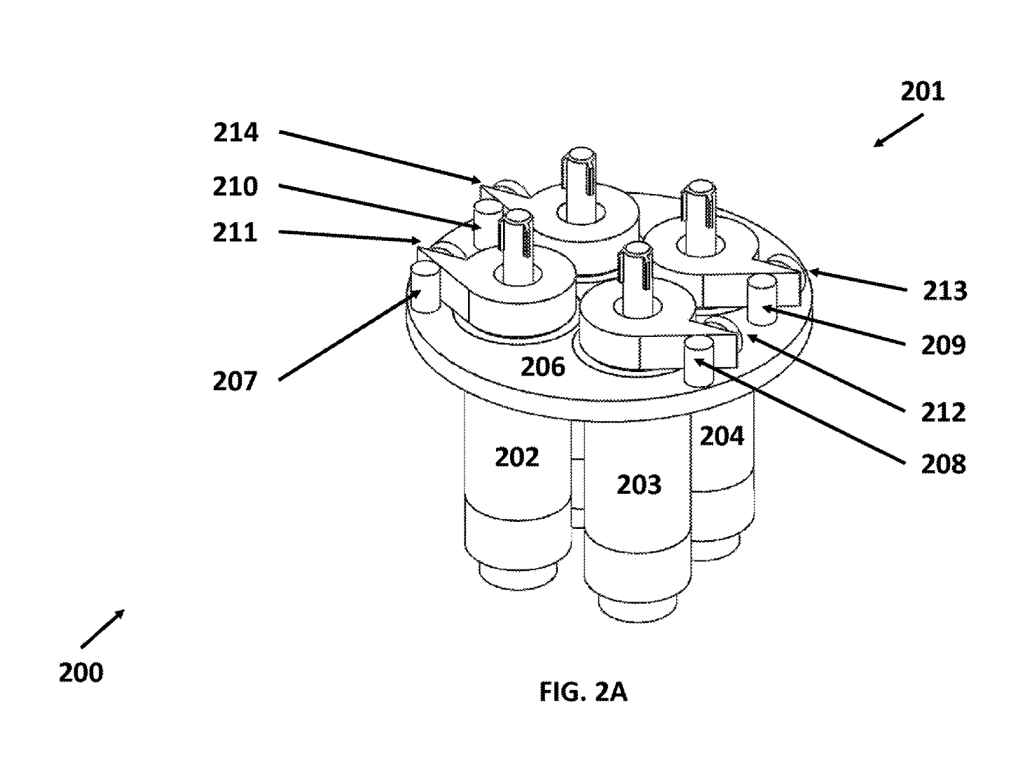

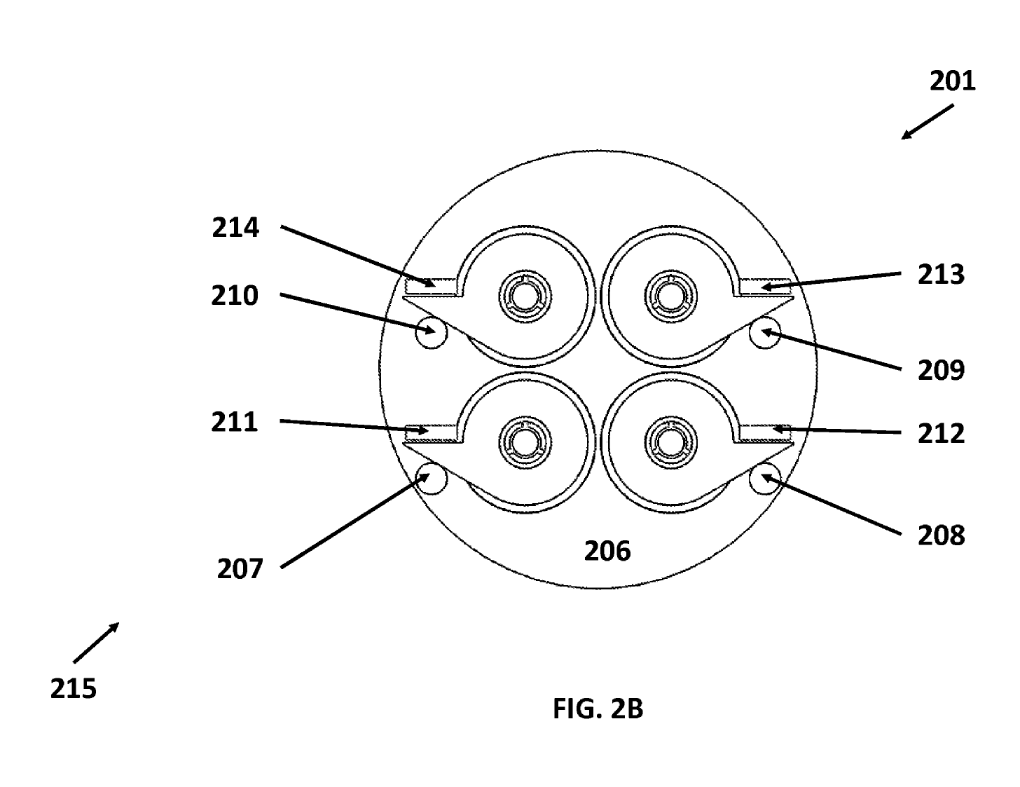

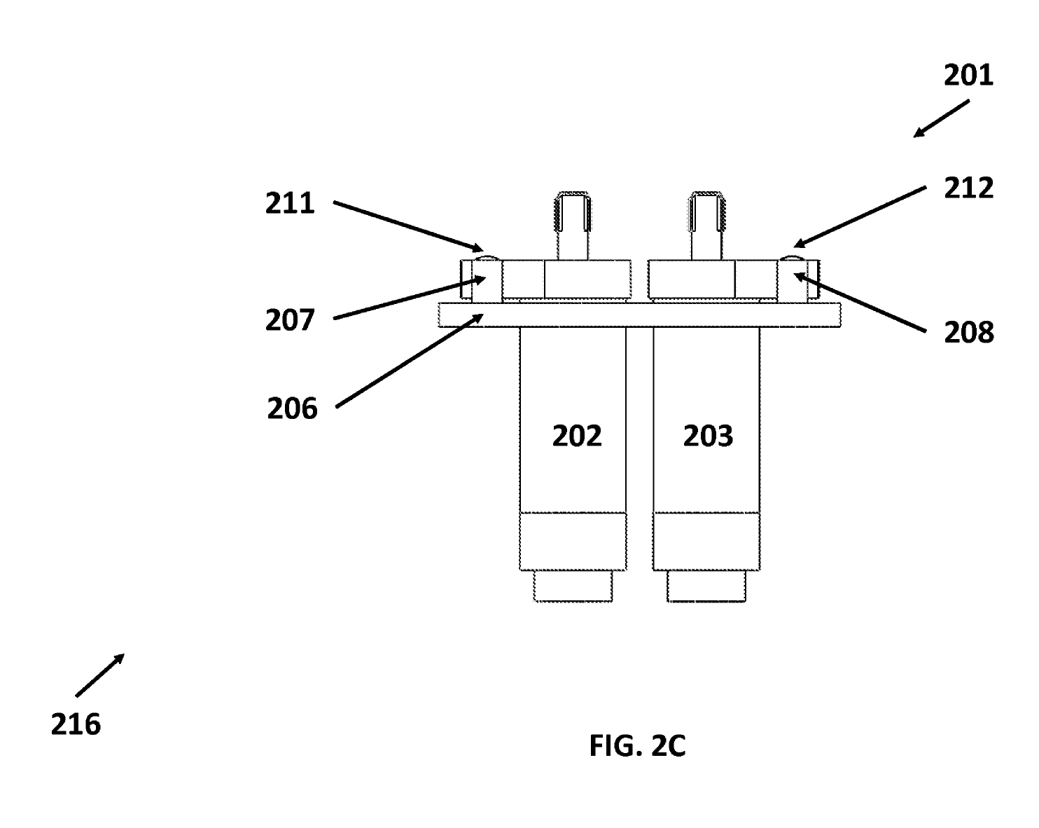

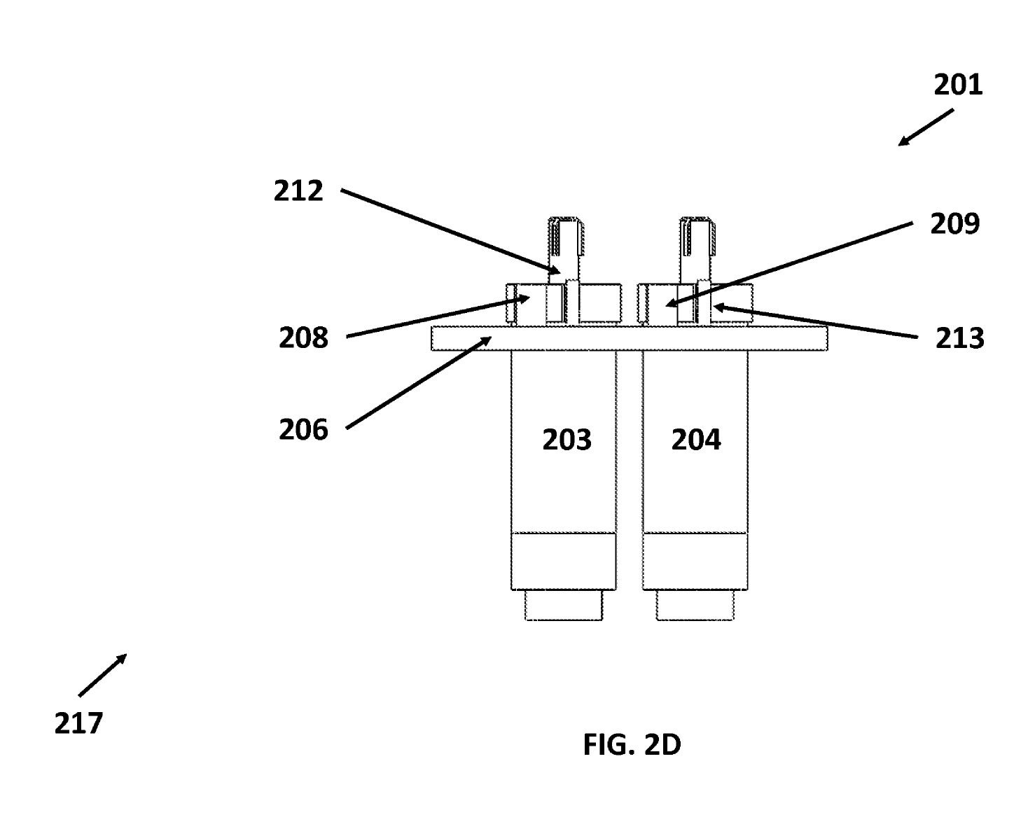

FIG. 2A illustrates a perspective view of a plurality of drive units as configured to be used in an instrument device manipulator, consistent with one embodiment. As shown in isometric view 200, four drive units, such as drive unit 202, drive unit 203, drive unit 204, and drive unit 205 (obscured by drive unit 202), each including components substantially disclosed in FIGS. 1A-1C, may be arranged in parallel fashion within an instrument device manipulator 201 such that their gear head shafts are parallel as well.

The drive units 202, 203, 204, and 205, may be held in place in parallel fashion by a drive base 206 that is coupled to the respective gear head and/or the motor units of the coupled drive units. Even though the drive base 206 is shown to be circular in FIG. 2A, the base may be any variety of shapes to accommodate the structure and/or shape of the instrument device manipulator.

As shown in view 200, the force sensors and stoppers are organized and arranged on base 206 to avoid touching, colliding, or conflicting each other and, thus, provide incorrect force measurements. For example, force sensors 212 and 213 are arranged to detect counter-clockwise reactive force resulting from clockwise motion in their respective gear head shafts. Accordingly, the stoppers 208 and 209 are arranged to prevent reactive motion in a clockwise direction and thus prevent the respective beam elements from hitting each other. In similar fashion, force sensors 207 and 210 are arranged to detect counter-clockwise reactive force while stoppers 211 and 214 are arranged to prevent clockwise motion and thus collision from the respective beam elements.

Within a robotic system, the instrument device manipulator may receive control signals from a controller that actuates the motor units to generate an output torque to control the attached instruments. The force sensors on each drive unit may also be configured to communicate the magnitude of the force detected, thus measuring the torque generated by the motor units, allowing feedback to the controller regarding the resulting torque generated by the motor units in the instrument device manipulator. Using the feedback, the controller may provide the proper control signal to the drive units to increase, decrease or maintain torque.

FIG. 2B illustrates a top view of an instrument device manipulator from FIG. 2A, consistent with one embodiment. As shown in top view 215, viewed from above instrument device manipulator 201, the respective beam elements, stoppers, and force sensors relative to the drive base 206 are arranged and fixed in position to avoid collisions resulting from reactive torque in the output shafts. FIGS. 2C and 2D illustrate alternative side perspectives on the instrument device manipulator from FIGS. 2A and 2B. In frontal view 216 from FIG. 2C illustrates the parallel alignment of drive units 202 and 203 and the relative arrangement of stoppers 207 and 208 and sensors 211 and 212 (both partially obscured). In side view 217 from FIG. 2D, the parallel alignment of drive units 203 and 204 and the relative arrangement of stoppers 207 and 208 and sensors 211 and 212 (both partially obscured).

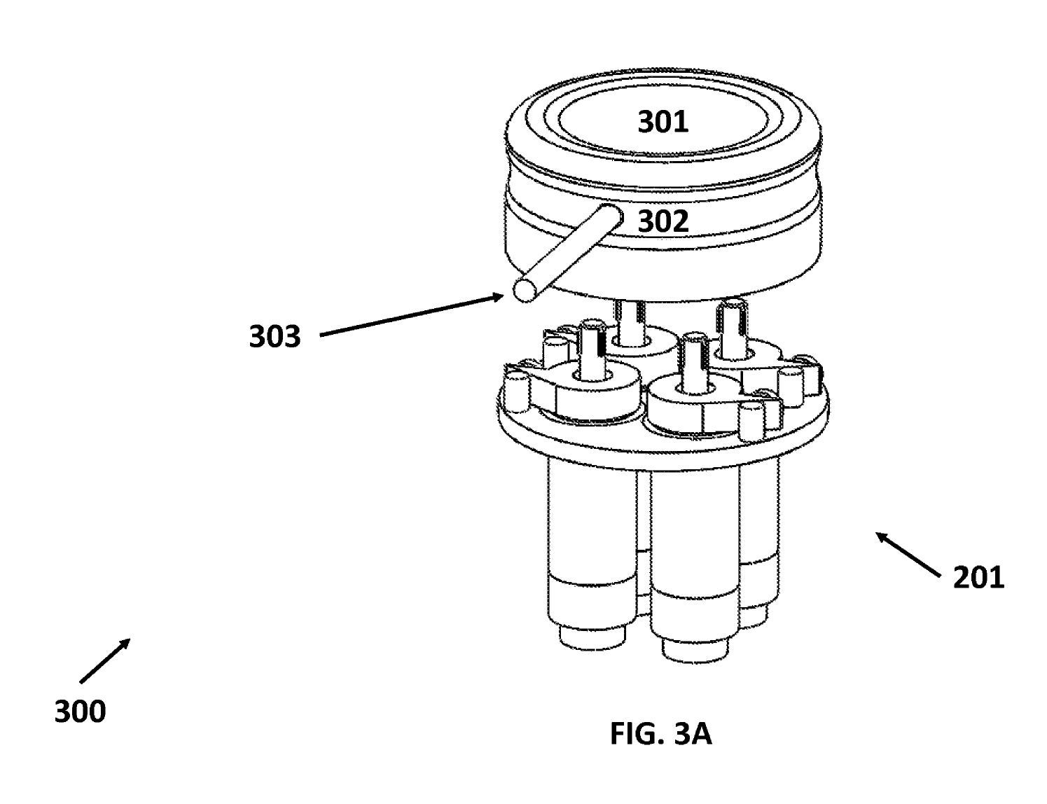

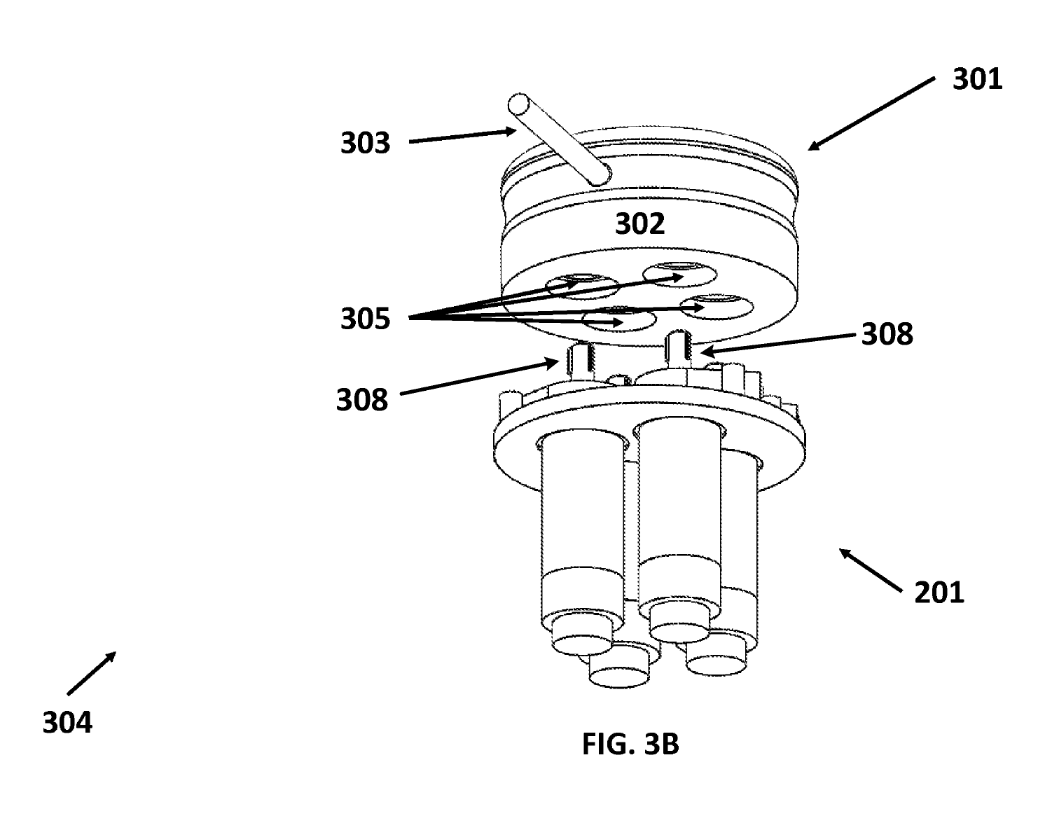

FIG. 3A illustrates one view of the alignment of an instrument device manipulator in combination with an associated instrument, consistent with one embodiment. As shown in isometric view 300, instrument driver 201 (shown without an outer skin or shell) may be configured to interface with an instrument 301. In some embodiments, the instrument interface may occur through a removable sterile adapter that may be cleansed. Instrument 301 may generally include an instrument base 302 and an elongated body 303, such as a catheter or endoscope, which is designed to be robotically actuated or robotically articulated for either endoscopic and laparoscopic procedures within a patient. Instrument 301 may be architected, constructed, and used in operative methods as disclosed in the aforementioned patents.

FIG. 3B illustrates another view of the alignment of an instrument device manipulator in combination with an associated instrument, consistent with one embodiment. As shown in view 304, the arrangement of parallel gear head shafts 308 in instrument device manipulator 201 allows for easy interfacing with associated instrument 301 and/or a sterile boundary interface. The parallel gear head shafts may be configured to align with ports 305 that each include a rotatable body, such as a pulley or spool, that may be fixedly coupled to a tendon or some other elongated member, such as a pull wire or cable, to actuate the elongated body 303. Given the four ports 305 in instrument base 302, instrument 301 provides for four separate means of actuation that be used to articulate the elongated body 303 with multiple degrees of freedom. The operative details of tendon actuation to actuate the elongated body may be consistent with the devices and methods disclosed in the aforementioned patents.

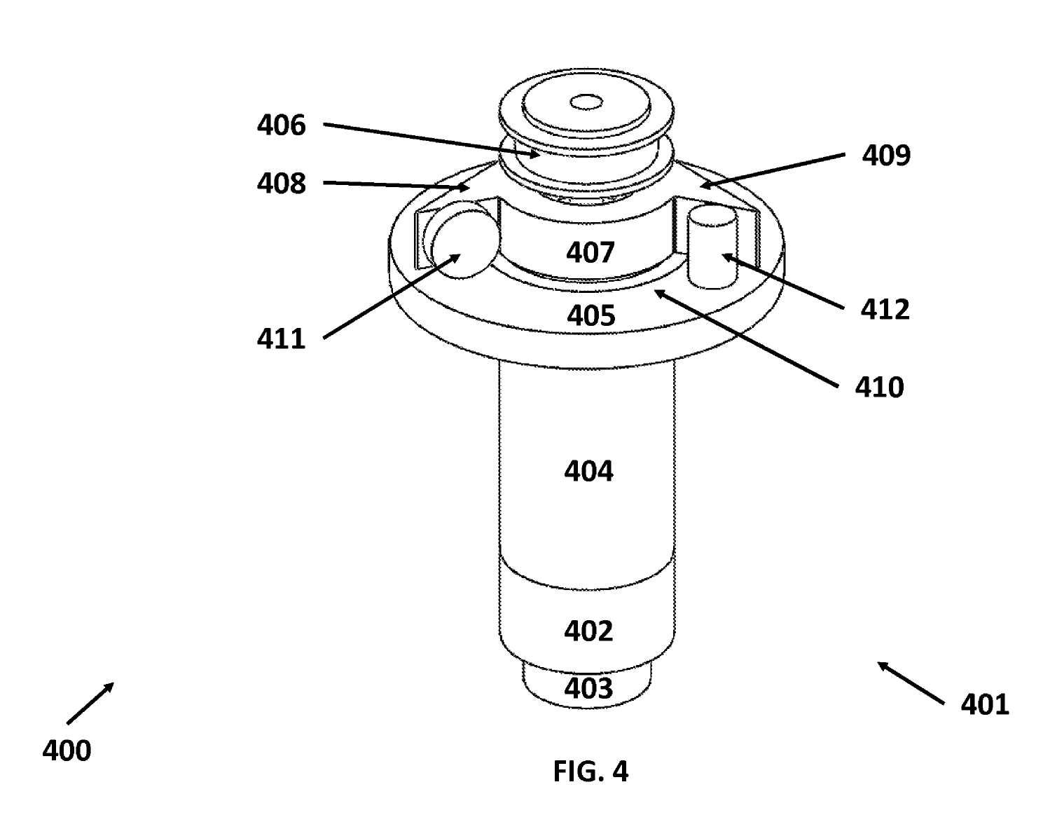

FIG. 4 illustrates an instrument drive mechanism that incorporates a torque sensing mechanism, consistent with one embodiment. As shown in isometric view 400, a drive unit 401 may generally include a motor unit 402, a rotary encoder 403, a gear head 404, a drive base 405, and an output pulley 406. Similar to the motor units disclosed in the aforementioned patents, the motor unit 402 may include a motor, such as a brushed or brushless motor or other electric motor.

Like the embodiments disclosed before, rotary encoder 403 monitors and measures the angular speed of the driveshaft of motor unit 402. As in the previous embodiments, rotary encoder 403 may be a redundant rotary encoder.

As in earlier embodiments, drive base 405 may include a bearing (410) that may be any type of low-friction bearing, such as a ball bearing and a roller bearing. In the alternative, the bearing in drive base 405 may be switched for a bushing or any other mechanical component that allows for angular motion. In some embodiments, the gear head 404 may be attached to the motor 402 to increase torque of the motor output, at the cost of the rotational speed. The increased torque generated by gear head 404 may be transmitted into a gear head shaft and output pulley 406.

In response to a control signal from a robotic controller, the torque generated by the motor 402 may be transmitted to output pulley 406 through a shaft coupled to the rotor of motor 402. Tension sensing apparatus 405 may generally include rotary component 407 and a first beam element 408 and a second beam element 409 coupled to different radial locations on rotary component 407. Designed to wrap around the rotor of motor unit 402, the rotary component 407 may be configured to freely rotate in the counter-clockwise direction in response to clockwise angular motion (and thus torque) of the rotor motor unit 402 and output pulley 406. In some embodiments, the rotary component 407 may be coupled to reflect and mirror angular motion in gear head 404, motor 402 and/or encoder 403 that results from rotating the rotor of motor 402 and gear head shaft (not shown). While rotary component 407 may be coupled to drive base 405, which is fixedly coupled to gear head 404, the rotary component 407 may be configured to freely rotate on a circular interface with a plurality of ball bearings (410) on drive base 405. The use of ball bearings is intended to provide a frictionless or near-frictionless path, such as in a bushing, for the rotary component 407 to move. In some embodiments, the circular interface may be co-axial with the rotor of the motor unit 402 and the output pulley 406, resulting a co-axial path for rotation of the rotary component 407 around the rotor of motor unit 402. In the absence of a gear head 404, the drive base 405 may be coupled to the motor 402 as a motor mount through a bearing interface.

The orientation of the coupling of the rotary component 407, the first beam element 408, and the second beam element 409, provides that the counter-clockwise torque generated by the rotary component 407 in response to clockwise torque from the rotor of motor 402 and output pulley 406 is detected by force sensor 411. Although the first beam element 408, and the second beam element 409 are orthogonal and perpendicular to the axial length of the rotary component, beam elements in other embodiments may oriented in other directions based on the position and location of their respective force sensors and stoppers.

In practice, clockwise angular motion of the rotor of motor 402 and output pulley 406 results in the generation of a proportional reactive counter-clockwise motion for the rotary component 407 as it rotates on ball bearings in the drive base 405. As the first beam element 408 is coupled to the rotary component 407, its direction of travel around the axis of the rotary component 407 would collide with the force sensor 411. As the force sensor 411 is designed to measure the force from the impact of first beam component 408, it may generate a signal that represents the magnitude of the force of that impact. This signal may be interpreted by a remotely-located robotic controller as a means of device feedback. Force sensor 411 may be any sensor that can detect applied force from the beam element 408, such as a load cell, piezoresistive device, a piezoelectric device, or a strain gauge.

In contrast to the force sensor, the stopper 412 is intended to limit the range of motion of the rotary component 407 and the first beam element 408. Where space is limited, the use of stopper 412 prevents beam element 408 from colliding with other components and generating inaccurate force readings at sensor 411 by “stopping” angular motion of beam element 409. In contrast to the embodiment of FIG. 1, in the embodiment of FIG. 4, the stopper 412 is positioned at a greater distance from the force sensor 411, and a second beam element (beam element 409) contacts the stopper 412 rather than the same beam element that contacts the force sensor 411 (beam element 408). In some embodiments, the stopper may be replaced by a second force sensor, allowing for the detection of angular force in both the clockwise and counter-clockwise directions.

Drive base 405 may also stabilize drive unit 401 within an instrument drive mechanism. In some embodiments, the drive base may be constructed from aluminum to reduce weight. In some embodiments, the motor unit 402 may be coupled to a gear head 404 to increase torque of the motor output, at the cost of the rotational speed. The increased torque generated by the gear head 404 may be transmitted to output pulley 406. The output pulley 406 may be configured to transmit the torque and angular motion of the rotor of motor unit 402 to belt transmission or some other form of gear train transmission. The drive base 405 or other potential motor mount may be to an exterior shell, electronic components, or other mechanisms within an instrument drive mechanism.

As with the earlier embodiments, the stopper 412, force sensor 411, and drive base 405 may be a fixed relative to each other. For example, they may be anchored or coupled to a single rigid frame, base, or mount. In addition, force sensor 411 may be “pre-loaded” by positioning the stopper against the beam element 408, such that the force sensor 411 detects a force measurement, e.g., a “baseline”, even when there is no angular motion in the output pulley 406. This allows for a single force sensor to detect angular motion in both clockwise and counter-clockwise directions.

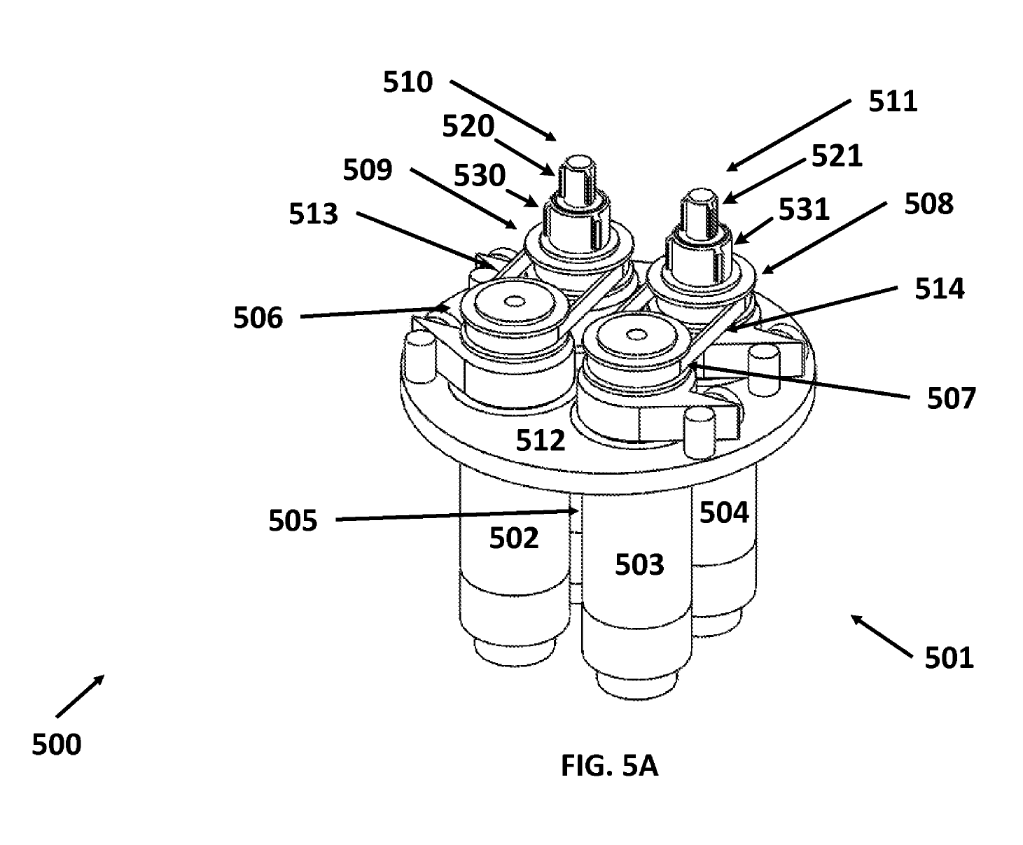

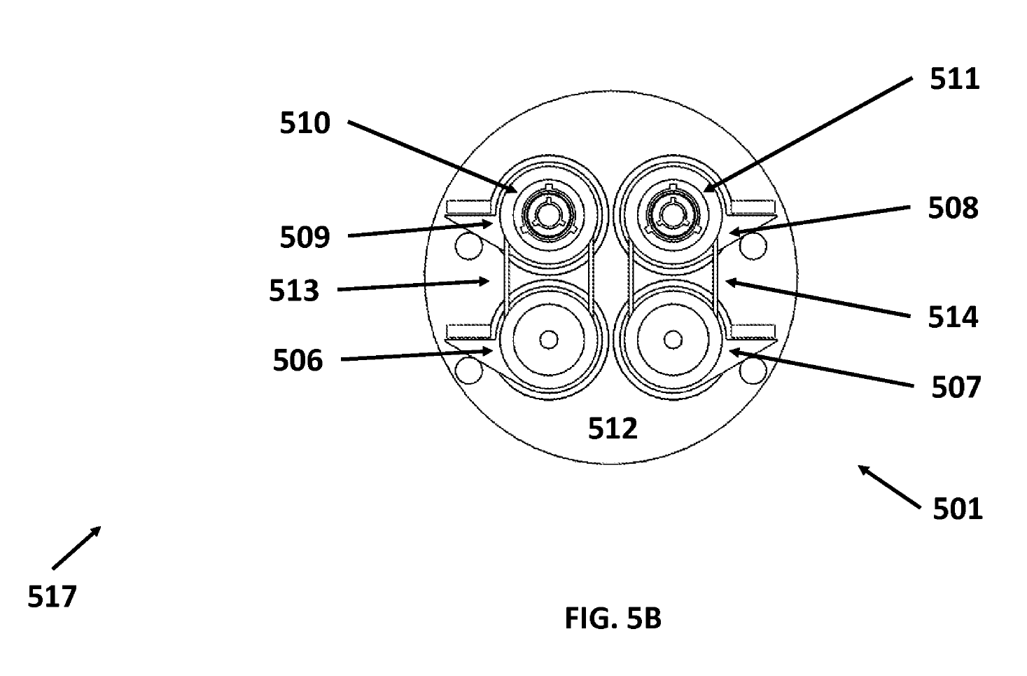

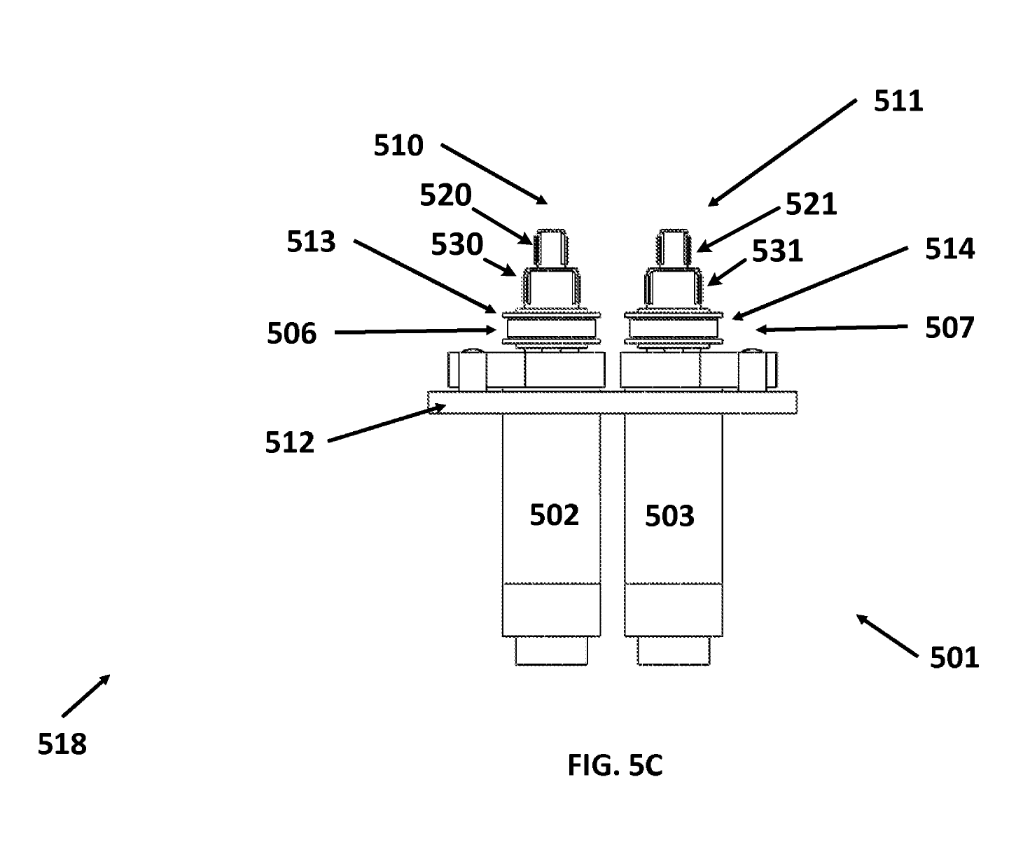

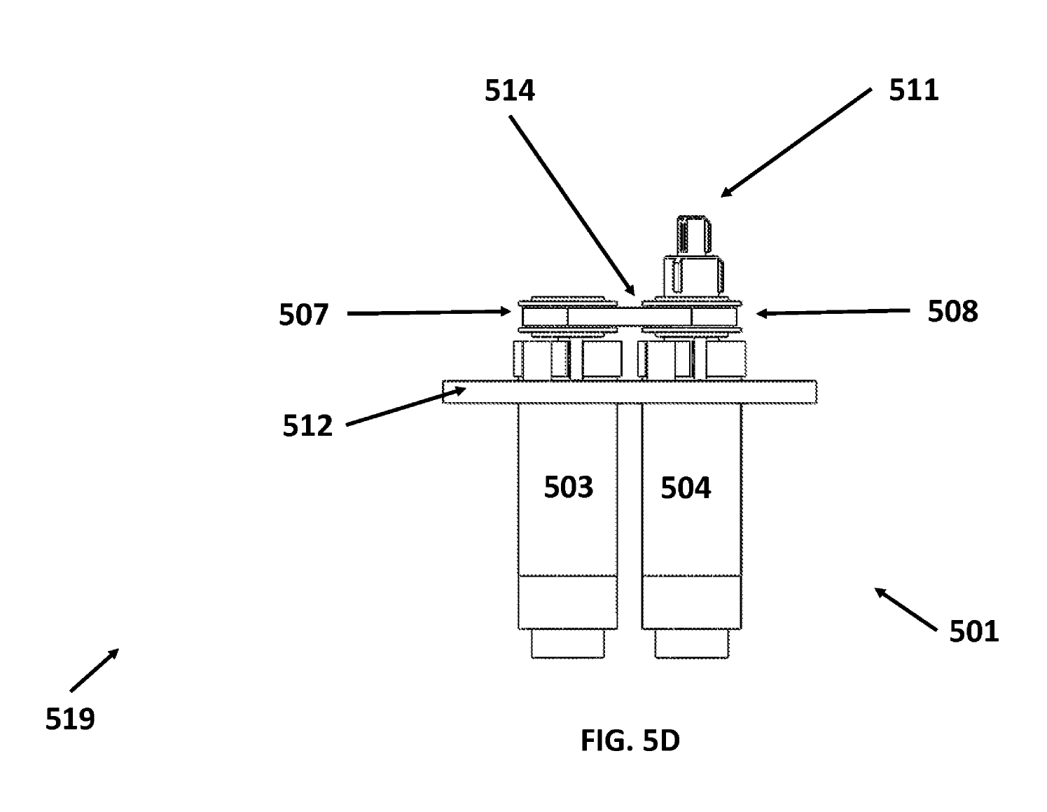

FIG. 5A illustrates the incorporation of a drive unit with a bi-directional torque sensing mechanism into an instrument device manipulator with co-axial output shafts, consistent with one embodiment. As shown in isometric view 500, instrument device manipulator 501 may include four drive units (502, 503, 504, 505) that incorporate output pulleys (506, 507) and input pulleys (508, 509) to generate angular force on a pair of axial output shafts (510 and 511). The four drive units may be held in place by a single drive base 512 that couples drive units 502, 503, 504, 505 together through separate ball bearing interfaces that allow the drive units to independently rotate in response to rotor rotation in each corresponding motor unit.

Within instrument device manipulator, both drive units 502 and 503 are configured very similarly to drive unit 401 from FIG. 4, wherein their motor units are configured to generate torque in output pulleys 506 and 507 in response to a control signal. Conversely, the rotary components for each drive unit are respectively configured to reactively rotate and interact with force sensors mounted on the drive base 512. Similar to the force sensors and stoppers in instrument device manipulator 201 from FIG. 2A, the force sensors and stoppers in instrument device manipulator 501 are fixedly positioned such that they do not conflict and prevent collisions from their respective beam elements.

Output pulleys 506 and 507, belts 513 and 514, and input pulleys 508 and 509 form a drivetrain. Output pulleys 506 and 507 are configured to transmit angular motion to belts 513 and 514 respectively. Belts 513 and 514 respectively transmit the angular motion from output pulleys 506 and 507 to input pulleys 509 and 508 respectively. In some embodiments, alternative drivetrains may be used, such as an alternative gear train or other transmission means.

Each coaxial output shaft 510 and 511, located atop of drive units 505 and 504 respectively, includes an inner shaft 520, 521 and an outer shaft 530, 531, both configured to rotate independent of the other through the use of a low friction interface, such as a ball bearing interface. The inner shafts 520, 521, the shafts with the smaller diameters, may be driven by the rotor of the motor unit, such as the rotors of motor units in drive units 505 and 504. In contrast, the outer shafts 530, 531, the shafts with the larger diameters, may be driven by the corresponding input pulley that is coupled to the outer shaft. For example, the outer shaft of co-axial output shaft 511 is driven by input pulley 508, which receives torque from output pulley 507 through belt 514. Thus, the outer shafts' 530 and 531 rotations reflect the angular motion of the output pulleys 506 and 507, respectively.

FIG. 5B illustrates the incorporation of a drive unit with a bi-directional torque sensing mechanism into an instrument device manipulator with co-axial output shafts from an another perspective, consistent with one embodiment. As shown in overhead view 517, drive base 512 of instrument device manipulator 501 couples four drive units through ball bearing interfaces that incorporate output pulleys (506, 507) that drive input pulleys (508, 509) to generate angular force on a pair of coaxial output shafts (510 and 511). As discussed earlier, belts 513 and 514 may transmit torque from output shafts 506 and 507 to input shafts 509 and 508 respectively. Moreover, coaxial output shafts 510 and 511 incorporate both an inner shaft with a smaller diameter, and an outer shaft with a larger diameter, as visible in view 517 by the two sets of splines in each coaxial shaft 510 and 511. As discussed earlier, the inner shaft may be driven by the rotor of the motor unit within the drive unit, while the outer shaft may be driven by the drive train (belt) from the output pulley to the inner pulley.

FIG. 5C illustrates the incorporation of a drive unit with a bi-directional torque sensing mechanism into an instrument device manipulator with co-axial output shafts from an yet another alternative perspective, consistent with one embodiment. As shown in frontal view 518, the drive units within instrument device manipulator 501 are aligned in parallel and coupled by the drive base 512 through individually parallel ball bearings (not shown). The structure of the coaxial radial shafts 510 and 511, with their inner shafts 520 and 521 and outer shafts 530 and 531 of varying diameters, are also visible in this view 518. While output pulleys 506 and 507 are visible in this view, the input pulleys 508 and 509 are obscured.

FIG. 5D illustrates the incorporation of a drive unit with a bi-directional torque sensing mechanism into an instrument device manipulator with co-axial output shafts from yet another alternative perspective, consistent with one embodiment. As shown in side view 519, the drive units 503 and 504 within instrument device manipulator 501 are aligned in parallel and coupled by the drive base 512 through individually parallel ball bearings (not shown). The structure of the coaxial radial shaft 511, with its inner shaft and outer shaft of varying diameter, is also visible in this view 519. In addition, the transmission from output pulley 507 to input pulley 508 through belt 514 is also clearly visible in this view 519.

The aforementioned embodiments may be integrated into a larger robotic system, where a robotic controller may generate control signals to the appropriate drive units to generate an output torque to control the attached instruments. The force sensor on the drive units may also be configured to communicate the magnitude of the reactive force detected, thus indicating the proportional torque generated, allowing feedback to the controller. Using the feedback, the controller may provide the proper control signal to the drive units to increase, decrease or maintain the level of torque.

The aforementioned embodiments may be designed to configure an instrument device manipulator and interface with an instrument as part of a larger robotics platform such as those disclosed in the aforementioned patent applications that are incorporated by reference. For example, the drive unit may be configured to be incorporated into an instrument drive mechanism or an instrument device manipulator that is attached to the distal end of a robotic arm through a sterile interface, such as a drape. The driving elements may be shafts (male) or shaft receptacles (female) with spline interfaces to transfer rotational motion from the instrument drive mechanism to the instrument. As part of a larger robotics system, robotic control signals may be communicated from a remotely-located user interface, down the robotic arm, and to the instrument device manipulator to control the embodiment (drive units). Conversely, force measurements may be communicated back to the controller and user interface to properly issue future control signals.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Elements or components shown with any embodiment herein are exemplary for the specific embodiment and may be used on or in combination with other embodiments disclosed herein. While the described embodiments are susceptible to various modifications and alternative forms, specific examples thereof have been shown in the drawings and are herein described in detail. The description is not limited, however, to the particular forms or methods disclosed, but to the contrary, covers all modifications, equivalents and alternatives thereof.

What is claimed is:

1. A robotically-controlled drive unit configured to generate angular motion comprising:
an electric motor unit comprising a rotor that is configured to generate an output torque in response to a robotic control signal;
a beam element configured to generate a reactive torque in response to the output torque generated by the rotor, the beam element coupled to the electric motor unit and oriented perpendicularly to the longitudinal axis of the rotor; and
a force sensor configured to detect the reactive torque and communicate the magnitude of the reactive torque to a robotic controller.

2. The drive unit of claim 1, wherein the beam element is configured to rotate around the axis of the rotor using a ball bearing.

3. The drive unit of claim 1, further comprising a gear head coupled to the electric motor unit, wherein the gear head is configured to amplify the output torque generated by the rotor.

4. The drive unit of claim 3, further comprising a drive base that comprises a ball bearing configured to allow the beam element to move freely in response to the output torque.

5. The drive unit of claim 3, further comprising an output shaft operatively coupled to the gear head, the output shaft configured to transmit angular motion to a rotatable body intended to actuate a tendon.

6. The drive unit of claim 5, wherein the tendon is a component of an instrument configured for performing at least one of an endolumenal procedure and a laparoscopic procedure.

7. The drive unit of claim 5, wherein the output shaft is configured to rotate an elongated body along its longitudinal axis.

8. The drive unit of claim 1, wherein the force sensor comprises at least one of a load cell, piezoresistive device, a piezoelectric device, and a strain gauge.

9. The drive unit of claim 1, further comprising a stopper that is configured to keep the beam element from moving in the direction of the stopper, the stopper and the force sensor fixedly coupled to a drive base.

10. The drive unit of claim 9, wherein the stopper and the force sensor are positioned on opposite sides of the beam element.

11. The drive unit of claim 1, further comprising:
a second beam element coupled to the electric motor unit and oriented perpendicularly to the longitudinal axis of the rotor; and
a stopper configured to keep the second beam element from moving in the direction of the stopper, the stopper and the force sensor fixedly coupled to a drive base.

12. The drive unit of claim 1, wherein the drive unit is incorporated into an instrument device manipulator as part of a surgical robotics platform, the instrument device manipulator further comprising a second drive unit.

13. The drive unit of claim 12, wherein the second drive unit is operatively coupled to the drive unit, the second drive unit configured to transmit angular motion to the drive unit.

14. The drive unit of claim 13, wherein the instrument device manipulator further comprises a third drive unit and a fourth drive unit, the third drive unit operatively coupled to the fourth drive unit, the third drive unit configured to transmit angular motion to the fourth drive unit.

15. The drive unit of claim 13, the drive unit further comprising:
an inner shaft operatively coupled to the electric motor unit, wherein the output torque of the electric motor unit causes the inner shaft to rotate; and
an outer shaft operatively coupled to the second drive unit, wherein the angular motion from the second drive unit causes the outer shaft to rotate.

16. The drive unit of claim 9, wherein the drive unit is incorporated into an instrument device manipulator as part of a surgical robotics platform, the instrument device manipulator further comprising a second drive unit, the second drive unit comprising:
a second beam element;
a second force sensor; and
a second stopper configured to keep the second beam element from moving in the direction of the second stopper, the second stopper and the second force sensor fixedly coupled to the drive base and positioned relative to the drive unit to avoid conflicts between the force sensors and the beam elements of the drive unit and the second drive unit.

17. The drive unit of claim 1, wherein the robotic controller is remotely located and is configured to generate the robotic control signal.

18. The drive unit of claim 1, wherein the robotic controller is configured to generate the robotic control signal based on the magnitude of the reactive torque detected by the force sensor.

19. The drive unit of claim 1, further comprising a rotary encoder coupled to the electric motor unit and configured to detect angular motion of the rotor.

20. The drive unit of claim 1, further comprising a second force sensor, the force sensor and the second force sensor positioned on opposite sides of the beam element from each other.

21. The drive unit of claim 20, wherein the force sensor and the second force sensor are coupled to a drive base and fixed relative to each other.

22. The drive unit of claim 1, further comprising a first compression spring disposed between the beam element and the force sensor.

23. The drive unit of claim 22, further comprising a second compression spring disposed between the beam element and a stopper, the first compression spring and the second compression spring positioned on opposite sides of the beam element.

24. The drive unit of claim 9, wherein the stopper is positioned relative to the beam element such that the force sensor detects a non-zero reactive torque when there is no output torque generated by the electric motor unit.
